(19) United States
(12) Patent Application Publication
(10) Pub. No.: US 2020/0206013 A1
(43) Pub. Date: Jul. 2, 2020

(54) Method and apparatus for a customizable hernia compression with breathable substrate

(71) Applicant: George Dwayne Hirst, Melbourne Beach, FL (US)

(72) Inventor: George Dwayne Hirst, Melbourne Beach, FL (US)

(21) Appl. No.: 16/350,707

(22) Filed: Dec. 26, 2018

(51) Int. Cl. A61F 5/26; A61F 5/34

(52) U.S. Cl. CPC A61F 5/26; A61F 5/28; A61F 5/34

(57) ABSTRACT

A method and apparatus for a customizable, hernia compression with breathable substrate comprised of a one-piece, pelvic belt with built-in perineal strap(s) made of a breathable laminate material, such as but not limited to Airflex2™ liner and the like as well as an exterior made entirely of Velcro™ loop fabric. Said invention also having plurality of separate, planar, hooked, Velcro™ compression strips allowing a user to position and apply the precise amount of pressure at the precise angle to allow maximum hernia support. The invention also having two embodiments: one with a single, compression pad pocket and another with dual, compression pad pockets (for single and dual hernias). An object of the invention is to provide enhanced, user-based hernia compression support.

### CROSS-REFERENCE TO RELATED APPLICATIONS

This application claims the benefit of U.S. Provisional Patent Application No. 62/708,954, filed on Jan. 2nd, 2018.

### FIELD OF THE INVENTION

The present invention generally relates to hernias. More specifically, it relates to a method for allowing a user to establish custom, accurate hernia compression.

### BACKGROUND

The first hernia belts were called trusses and were first invented in the late 1800s. They were comprised of leather straps and metal springs and were not very comfortable. As elastomers and plastics became popular in the mid 1900s, lightweight, comfortable compression belts began replacing trusses. As Velcro™ emerged in the 1970s, belts became more form-fitting and could be worn discreetly under athletic clothes. Hernia belts have continued to evolve. U.S. Pat. No. 6,6227,19B1 granted to Slautterback et. al. disclosed a hernia compression apparatus built into underwear. U.S. Pat. No. 6,422,242 granted to Slautterback et. al. disclosed a hernia compression apparatus with a comprehensive Velcro™ system. U.S. Pat. No. 6,112,704 granted to Altafi teaches of an inguinal hernia belt having replaceable compression pads. U.S. Pat. No. 4,787,379 granted to Yeh disclosed a hernia belt having a flexible metal band as a compression patch. While these apparatus represent a vast improvement over trusses of the past, no patents were found that allow a user to customize their belt to their personal hernia support needs.

### SUMMARY OF THE INVENTION

The device herein disclosed and described provides a method for a user-defined, hernia compression apparatus with breathable substrate. An object of the invention is to allow a user, in conjunction with their doctor, to tailor compression points along a hernia belt to provide maximum support. While most hernias tend to occur in the lower torso, their exact location and size can vary for each person. A majority of belts currently available only provide flat patch support in these commonly occurring areas of the body. The invention in this disclosure includes several Velcro™ strips that can be positioned in an infinite number of ways on and around prepositioned hernia patches within a belt. The strips can also be overlapped with one another to enhance a flat compression patch and provide pinpoint support at various angles not possible with conventional belts. The invention also having two embodiments: a singular compression patch version and a dual compression patch version. Said single embodiment having one leg loop and the dual having tow leg loops.

Another object of the invention is to provide compression for a single as well as dual hernias in a single belt. In one embodiment, the belt comes with a single, pocket with an opening in the top to allow different thicknesses of padding to be swapped out depending on how much pressure the person needs in order to keep the hernia pushed in. In another embodiment, the belt comes with dual, pockets—one on both sides of the invention.

Another object of the invention is to provide a means to allow a user to vary the amount of direct pressure on a hernia. Hernias can protrude more or less at different times and depending on what an individual is doing at the time. Using the invention, a user can affix support strips in such a manner as to increase or decrease compression load on a patch at any given time. For example, if a hernia needs more compression strength, multiple support strips can be slung across the front of the belt accessing more Velcro™ surface area to provide greater stiffness to compression at the hernia in the front portion of the belt. Likewise a user may select to use shorter strips to engage less adhesion allowing for less compression.

Another object of the invention is to provide a means to allow a user to provide compression at various angles. Hernia protrusions can occur at various angles at various times, also depending on what a user is doing. The invention disclosed allows a user to affix support strips in such a manner as to pinpoint the exact angle of compression needed on that particular day depending on what activity will be taking place. For example, on one day a user may be operating a wheelbarrow that may cause a hernia to protrude in a downward manner and they can position support strips together that will provide an upward force. On another day the user may be laying on a paddle board that may cause a hernia to protrude in a lateral manner and they can position strips together that will provide a counter-force.

Another object of the invention is to enhance the strength of a hernia belt. All components of the apparatus are made of a water-resistant, form-fitting, breathable neoprene laminates. This material can compress a hernia and allow a user to comfortably participate in rigorous sports. Neoprene laminates are made of a nylon, rubber, spandex fusion that is stronger than conventional hernia belts made from elastic textiles and cotton fabrics.

Another object of the invention is to provide a means of enhancing hernia belt comfort. The invention has a slippery liner made of a breathable material to prevent rashes. This allows the invention to prevent the skin from chaffing while wearing a hernia compression apparatus under various compression loads. The exterior portion of the belt is made up entirely of Velcro™ loop fabric eliminating the need for additional hook layers and enhancing the streamlined-profile allowing for greater comfort therein.

Another object of the invention is to enhance the ease of use of a hernia belt. The single compression pad embodiment has a single attachment point (rectangular portion) as part of the belt and the dual compression pad embodiment has three attachment points (rectangular portions) as part of the belt. However, both embodiment points are built into the form of the invention making donning and doffing the belt much easier than conventional belts that have bulky, overlapping, multiple attachment points.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

The accompanying drawings, which are incorporated herein and form a part of the specification, illustrate some, but not the only or exclusive, examples of embodiments and/or features.

FIG. 1 showing a front and rear views of an embodiment the invention with single compression patch.

FIG. 2 showing a side view of the invention with Velcro™ strap.

FIG. 3 showing a side view of the invention with Velcro™ strap.

FIG. 4 showing a perspective view of an embodiment with a single compression pocket.

FIG. 5 showing a perspective view of an embodiment with a double compression pocket.

FIG. 6 showing the invention with support strips installed.

FIG. 7 showing a front and rear views of an embodiment the invention with double compression pocket.

Other aspects of the present invention shall be more readily understood when considered in conjunction with the accompanying drawings, and the following detailed description, neither of which should be considered limiting.

### DETAILED DESCRIPTION OF FIGURES

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation.

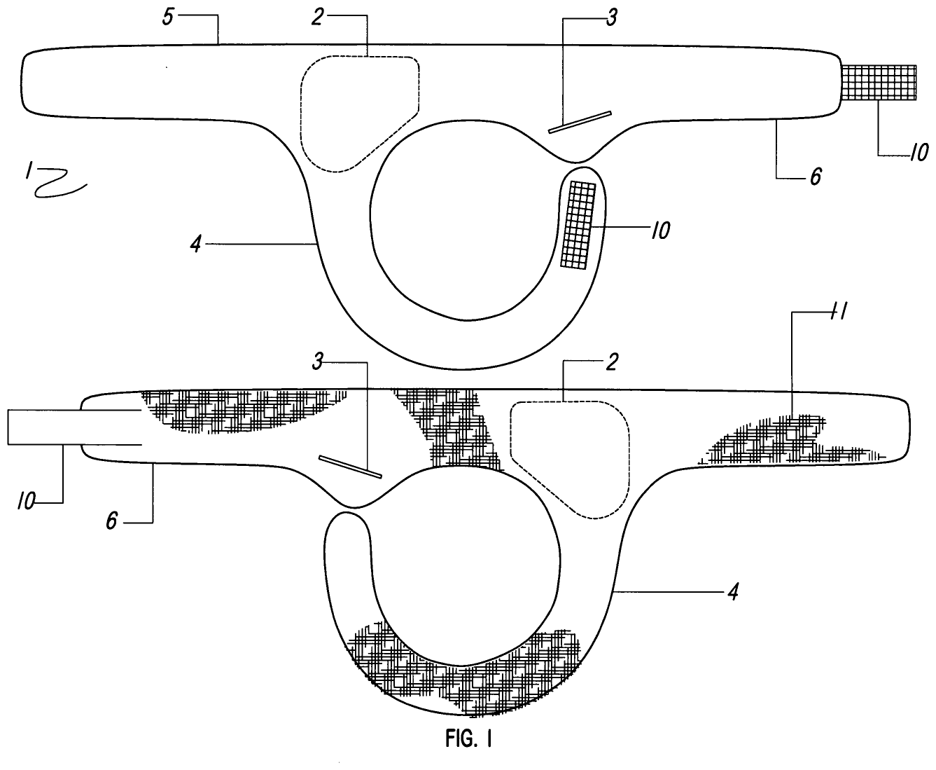

FIG. 1 showing a front and rear views of an embodiment of the invention 1 with one compression pocket 2 and having three parts. The first part being a rectangular-shaped belt 5, a second rectangular-shaped cummerbund 6 part extending from said belt 5 and a third loop 4 part protruding from the longitudinal axis of the aforementioned belt 5. All said parts having two walls of material therein. Said one wall facing outwardly of a user comprised of a loop material 11, such as but not limited to hook and loop Velcro™ thereon. Another wall facing inwardly to a user comprised of a breathable material, such as but not limited to Airflex2™ and the like. A rectangular slot 8 being disposed diagonally to the longitudinal axis of said belt configured to receive said loop 4. The aforementioned loop 4 and cumberbund 6 having a rectangular-shaped patch comprised of hook material 10, such as but not limited to Velrco™ thereon.

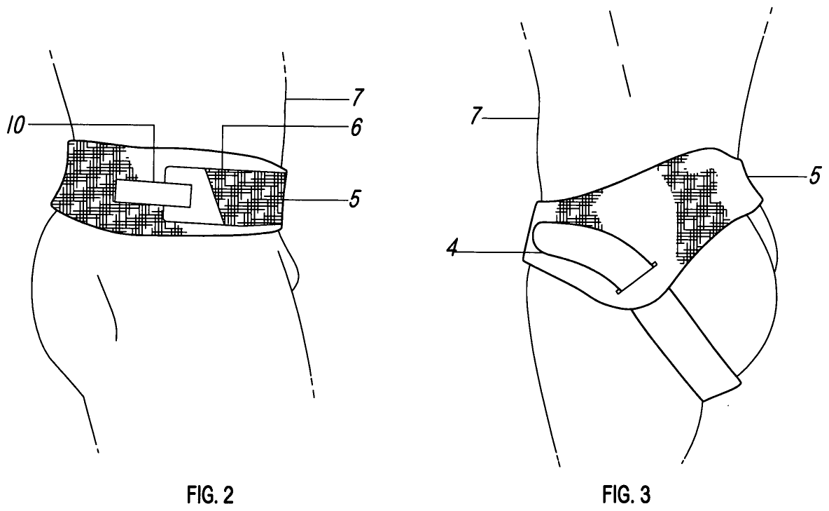

FIG. 2 showing a side view of the invention with said cummerbund 6 being releasable secured to belt 5 by means of said Velcro™ 10.

FIG. 3 showing a side view of the invention with said loop 4 passing through said slot 8 and also being selectively affixed to belt 5 with Velcro™ strap.

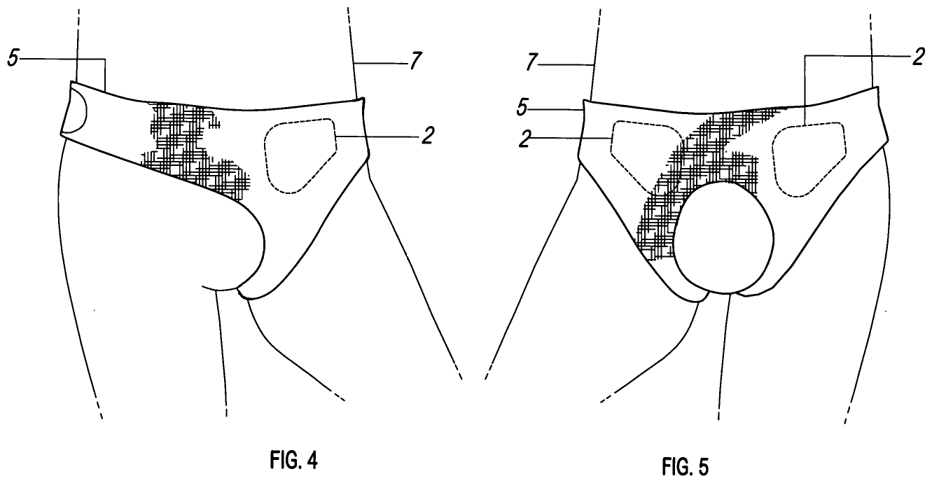

FIG. 4 showing a perspective view of an embodiment with a single compression pocket 2. Said compression pockets configured to receive a predetermined amount of padding such as but not limited to: neoprene foam squares, cotton swabs, and the like. Other embodiments may have a single pocket on either side of said belt 5.

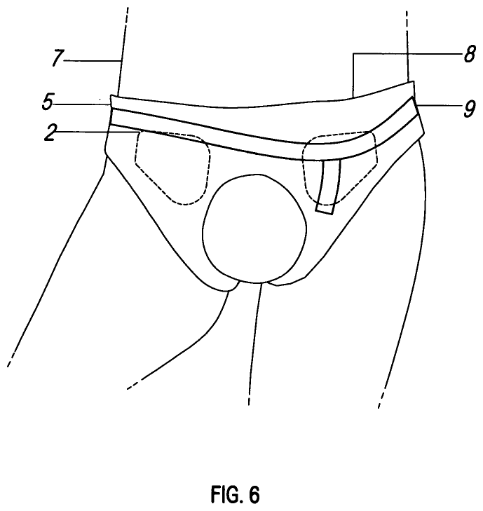

FIG. 5 showing a perspective view of an embodiment with a double compression pocket 2.

FIG. 6 showing the invention with support strips 9 installed at a plurality of locations along said belt 5.

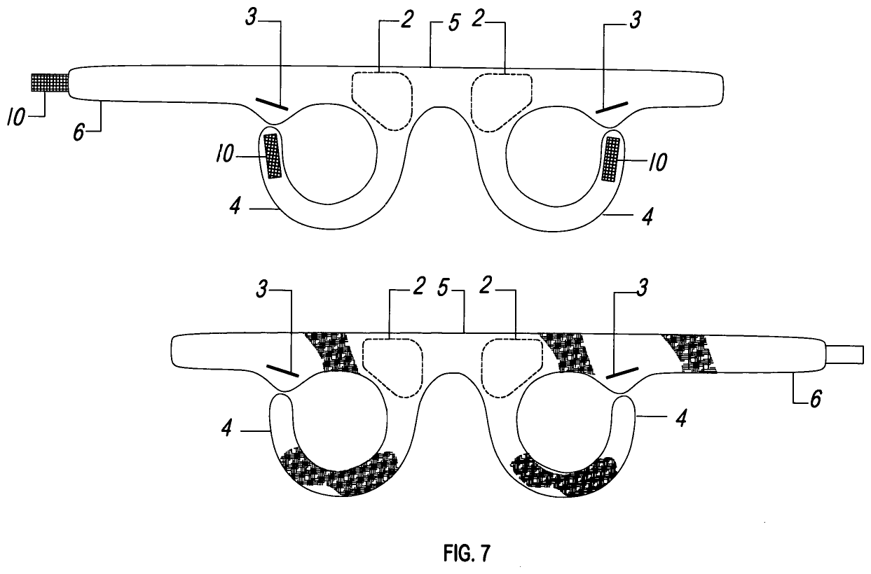

FIG. 7 showing a front and rear views of an embodiment the invention with double compression pockets 2. Said dual patch embodiment having two leg loops 4 thereon.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention.

What is claimed is:

1. A hernia belt system comprised of the following parts:
a.) a belt;
b.) pockets; and
c.) hook and loop strips.

2. A method comprising:
a.) supporting a hernia at a multitude of angles by means of selectively placing hook and loop strips at various locations on the hernia belt system.
c.) compressing a hernia using a plurality of pressures by means of selectively inserting materials of various thicknesses inside hernia belt pockets.
